(19) United States
(12) Patent Application Publication
(10) Pub. No.: US 2020/0282125 A1
(43) Pub. Date: Sep. 10, 2020

(54) INDIVIDUALIZED DIALYSIS WITH INLINE SENSOR

(71) Applicant: Fresenius Medical Care Holdings, Inc., Waltham, MA (US)

(72) Inventors: Shashikant Dattatraya Kalaskar, Layton, UT (US); Vivian Changna Wang, Farmington, UT (US)

(21) Appl. No.: 16/811,533

(22) Filed: Mar. 6, 2020

Related U.S. Application Data
(60) Provisional application No. 62/815,242, filed on Mar. 7, 2019.
(60) Provisional application No. 62/967,349, filed on Jan. 29, 2020.

(51) Int. Cl. A61M 1/16; A61M 1/34

(52) U.S. Cl. CPC A61M 1/1654; A61M 1/1605; A61M 2205/3306; A61M 1/3465; A61M 1/3441

(57) ABSTRACT

A method for adjustment of a dialysate during dialysis for a patient is provided. A patient undergoing a dialysis treatment, e.g., a hemodialysis (HD) treatment, can experience multiple physiological changes during the treatment. These can include change in blood volume as well as change in concentration of blood electrolytes. Blood electrolytes when taken out of their desired ranges can result in one or more health risks. The disclosure provides a way of avoiding those health risks by adjusting composition of dialysate during dialysis treatment such that blood electrolytes are maintained within their desired ranges.

### CROSS-REFERENCE TO RELATED APPLICATIONS

This patent application claims the benefit of U.S. Provisional Patent Application No. 62/967,349, filed Jan. 29, 2020, and U.S. Provisional Patent Application No. 62/815,242, filed Mar. 7, 2019, both of which are incorporated by reference herein in their entirety.

### BACKGROUND

Patients with kidney failure or partial kidney failure typically undergo hemodialysis treatment at hemodialysis treatment centers, clinics, or in the home. When healthy, kidneys maintain the body's internal equilibrium of water and minerals (e.g., sodium, potassium, chloride, calcium, phosphorous, magnesium, and sulfate). In hemodialysis, blood is taken from a patient through an intake needle (or catheter) which draws blood from an artery located in a specific access location (arm, thigh, subclavian, etc.). The blood is then pumped through extracorporeal tubing via a peristaltic or other pump, and then through a special filter called a dialyzer. The blood passes through the dialyzer in contact with an internal semipermeable membrane, typically in a countercurrent direction to the flow of a dialysate solution on the opposite side of the membrane. The dialyzer is intended to remove unwanted toxins such as urea, nitrogen, and potassium, as well as excess water from the blood by diffusion and/or convective transport, depending on the specific type of dialysis ordered. The dialyzed blood then flows out of the dialyzer via additional tubing and through a needle (or catheter) back into the patient.

During dialysis, an excess of electrolytes in the patient's blood may be lost. Also in some cases, dialysis may result in insufficient removal of electrolytes. For example, blood contains sodium ions (Na⁺), potassium ions (K⁺), and calcium ions (Ca²⁺). Too much sodium in the blood can contribute to the patient feeling an increase in thirst or can lead to hypertension. Losing too much sodium can lead to decline in blood volume, chest pain, nausea, vomiting, headache, and muscle cramps. Too much potassium in the blood can lead to muscle pain, weakness, and numbness. Losing too much potassium can lead to heart rhythm disturbances. Having too much calcium in the blood can lead to vascular calcification. Losing too much calcium can lead to bone disorders and/or uncontrollable secondary parathyroid hormone (PTH) secretion.

Electrolyte composition in the blood is a highly dynamic function, dependent on many physiological and nutritional inputs, and subject to significant variability between patients. In most dialysis settings, one or only a small number of dialysate compositions (i.e., “recipes”) is available to treat patients, regardless of individual variations in electrolyte profiles that exist between patients or even between the same patient on different days. This “one-size-fits-all” approach to treatment may be reasonable for the majority of patients, but some patients do not tolerate it well. Accordingly, a method and system for preparing a patient-specific dialysate would be advantageous, and one that can adapt to real-time changes in patient needs between and even during dialysis treatments.

### SUMMARY

An embodiment of the disclosure provides a method for adjustment of a dialysate during dialysis for a patient. The method comprises: subsequent to initiating dialysis for the patient, obtaining, by a controller and from an electrolyte sensor, a measurement of a concentration of an electrolyte in the patient's blood; determining, by the controller, whether the obtained measurement is within a predefined range; in response to determining that the measurement is not within the predefined range, determining, by the controller and based on the obtained measurement, at least one first adjustment value for adjusting a composition of the dialysate, wherein the composition of the dialysate is based on respective amounts of chemicals dispensed from a plurality of chemical sources; and controlling, by the controller and based on the at least one first adjustment value, a dispenser to adjust the composition of the dialysate during dialysis for the patient by changing one or more of the respective amounts of chemicals dispensed from the plurality of chemical sources.

In another embodiment of the disclosure, the method comprises controlling, based on the at least one first adjustment value, the dispenser to adjust the composition of the dialysate by providing one or more first instructions to direct the dispenser to adjust the composition of the dialysate. Additionally, the method further comprises: subsequent to adjusting the composition of the dialysate during dialysis based on the at least one first adjustment value, obtaining, by the controller, a second measurement of the concentration of the electrolyte in the patient's blood from the one or more electrolyte sensors; determining, by the controller, whether the at least one first adjustment value caused the second measurement to be within the predefined range; and in response to determining that the second measurement is not within the predefined range, providing one or more second instructions to the dispenser to adjust the composition of the dialysate during dialysis based on the second measurement.

In another embodiment of the disclosure, the method further comprises in response to determining that the second measurement is within the predefined range, maintaining the composition of the dialysate during dialysis.

In another embodiment of the disclosure, the method further comprises: obtaining, by the controller, a second measurement of a second concentration of a second electrolyte in the patient's blood, wherein the second electrolyte and the first electrolyte are different electrolytes; determining, by the controller, whether the second measurement is within a second predefined range; in response to determining that the second measurement is not within the second predefined range, determining, by the controller and based on the second measurement, at least one second adjustment value for adjusting the composition of the dialysate, and wherein controlling the dispenser to adjust the composition of the dialysate is based on the at least one first adjustment value and the at least one second adjustment value.

In another embodiment of the disclosure, the method comprises controlling the dispenser to adjust the composition of the dialysate by generating, by the controller, actuating signals for changing the composition of the dialysate during dialysis based on the at least one first adjustment value; and providing, by the controller, the actuating signals to one or more actuators of the dispenser to change proportions of the respective amounts of chemicals dispensed from the plurality of chemical sources.

In another embodiment of the disclosure, the method comprises: based on determining the at least one first adjustment value the electrolyte is an increase, generating a first actuating signal for dispensing a higher proportion of a respective chemical of a respective chemical source of the plurality of chemical sources; and based on determining the at least one first adjustment value the electrolyte is a decrease, generating a second actuating signal for dispensing a lower proportion of the respective chemical of the respective chemical source of the plurality of chemical sources.

In another embodiment of the disclosure, the method further indicates each actuating signal of the actuating signals is encoded as: a reduction in the number of electrical pulses provided to one actuator of the one or more actuators, an increase in the number of electrical pulses provided to one actuator of the one or more actuators, a reduction in the number of electrical pulses provided to all but one actuator of the one or more actuators, or an increase in the number of electrical pulses provided to all but one actuator of the one or more actuators.

In another embodiment of the disclosure, the method further comprises: receiving, by the controller, a fresh dialysate signal at time t_{f}, the fresh dialysate signal indicating that the dialysate is mixed and ready for use; and determining, by the controller and based on a flowrate of the dialysate and a volume of the dialysate, a time tₘₐₓ indicating a maximum amount of time after t_{f} to adjust the composition of the dialysate.

In another embodiment of the disclosure, the electrolyte sensor is an optical sensor.

In another embodiment of the disclosure, the electrolyte sensor is the NMR sensor, and wherein the NMR sensor is configured to obtain a real-time sodium concentration, a real-time potassium concentration, or a real-time phosphorous concentration.

In another embodiment of the disclosure, the electrolyte sensor is located upstream of a dialyzer and configured to interface with a tubing upstream of the dialyzer.

In another embodiment of the disclosure, the electrolyte sensor is located downstream of a dialyzer and configured to interface with a tubing downstream of the dialyzer.

In another embodiment of the disclosure, the method comprises determining a dialysate recipe based on the patient, and wherein determining the at least one first adjustment value for adjusting the composition of the dialysate is based on the dialysate recipe.

In another embodiment of the disclosure, the method comprises determining the dialysate recipe based on historical trend analysis from the patient's previous dialysis treatments.

In another embodiment of the disclosure, the method comprises controlling the dispenser to adjust the composition of the dialysate comprises providing one or more first instructions to the dispenser to adjust the composition of the dialysate. The method further comprises: subsequent to providing the one or more first instructions, obtaining, by the controller, a second measurement of the concentration of the electrolyte from the one or more electrolyte sensors; and determining an effectiveness of the dialysate recipe based on the second measurement.

In another embodiment of the disclosure, the method comprises determining the effectiveness of the dialysate recipe based on whether the second measurement is within the predefined range. The method further comprises based on determining the second measurement is not within the predefined range, selecting a new dialysate recipe; determining at least one second adjustment value for adjusting the composition of the dialysate based on the new dialysate recipe; and providing, to the dispenser of the electrolyte composition monitor, one or more second instructions to adjust the composition of the dialysate during dialysis based on the at least one second adjustment value.

In another embodiment of the disclosure, the method further comprises based on determining the dialysis for the dialysis patient has concluded, storing the dialysate recipe and the determined effectiveness of the dialysate recipe in memory.

Another embodiment of the disclosure provides a non-transitory computer-readable medium having processor-executable instructions stored thereon for adjustment of a dialysate during dialysis for a patient. The processor-executable instructions, when executed, facilitate: subsequent to initiating dialysis for the patient, obtaining, from an electrolyte sensor, a measurement of a concentration of an electrolyte in the patient's blood; determining whether the obtained measurement is within a predefined range; in response to determining that the measurement is not within the predefined range, determining, based on the obtained measurement, at least one first adjustment value for adjusting a composition of the dialysate, wherein the composition of the dialysate is based on respective amounts of chemicals dispensed from a plurality of chemical sources; and controlling, based on the at least one first adjustment value, a dispenser to adjust the composition of the dialysate during dialysis for the patient by changing one or more of the respective amounts of chemicals dispensed from the plurality of chemical sources.

Another embodiment of the disclosure provides a system for adjustment of a dialysate during dialysis for a patient. The system comprises an electrolyte sensor configured to measurement a measurement of a concentration of an electrolyte in the patient's blood; and an electrolyte composition monitor. The electrolyte composition monitor comprises a controller and a dispenser. The controller is configured to: subsequent to initiating dialysis for the patient, obtain, from the electrolyte sensor, the measurement of the concentration of the electrolyte in the patient's blood; determine whether the obtained measurement is within a predefined range; in response to determining that the measurement is not within the predefined range, determine, based on the obtained measurement, at least one first adjustment value for adjusting a composition of the dialysate, wherein the composition of the dialysate is based on respective amounts of chemicals dispensed from a plurality of chemical sources; and provide, based on the at least one first adjustment value, one or more first instructions to a dispenser to adjust the composition of the dialysate during dialysis for the patient by changing one or more of the respective amounts of chemicals dispensed from the plurality of chemical sources. The dispenser is configured to: adjust the composition of the dialysate during dialysis based on the one or more first instructions.

In another embodiment of the disclosure, the controller is further configured to: subsequent to adjusting the composition of the dialysate during dialysis based on the at least one first adjustment value, obtain a second measurement of the concentration of the electrolyte in the patient's blood from the one or more electrolyte sensors; determine whether the at least one first adjustment value caused the second measurement to be within the predefined range; and in response to determining that the second measurement is not within the predefined range, provide one or more second instructions to the dispenser to adjust the composition of the dialysate during dialysis based on the second measurement.

### BRIEF DESCRIPTION OF THE DRAWINGS

FIG. 1 illustrates a front perspective view of a hemodialysis system that includes an electrolyte composition monitor according to some embodiments of the disclosure;

FIG. 2 is a block diagram illustrating use of an electrolyte composition monitor with a patient, according to embodiments of the disclosure;

FIG. 3 is a flow diagram for managing electrolytes in blood of a dialysis patient during dialysis, according to an embodiment of the disclosure;

FIG. 4 is an example timeline for managing electrolytes in blood of a dialysis patient during dialysis, according to an embodiment of the disclosure;

FIG. 5 is a flow diagram for determining individualized dialysate recipes or prescriptions for a patient;

FIG. 6 is a block diagram of an example computer system; and

FIGS. 7a and 7b are graphical representations of real-time electrolyte concentration measurements using an NMR sensor.

### DETAILED DESCRIPTION

During dialysis, an electrolyte composition monitor according to embodiments of the disclosure can employ a dialysate mixing system to make an amount of dialysate on demand using, among other things, a plurality of chemical concentrates. The dialysate generated will have a formula, recipe, or prescription that differs from dialysate previously used during dialysis. The dialysate's formula will thus be adjusted during dialysis based on the electrolyte composition monitor detecting an elevated or depressed level of one or more electrolytes in a patient's blood during dialysis.

In an embodiment, the dialysate used during dialysis is made in batches. Each batch follows a prescription, formula, or recipe chosen by the electrolyte composition monitor based on receiving electrolyte concentration levels from the patient's blood. The electrolyte composition monitor may continually adjust a next dialysate batch's recipe and task its dialysate mixing system to follow the prescribed recipe. For example, the dialysate mixing system may receive a recipe indicating particular chemical constituents and amounts of each chemical constituent to be included in the dialysate. Based on the prescription, the dialysate mixing system can determine, for example, a number of tablets, mass of powder, or volume of concentrated electrolyte solution required for each chemical constituent. Tablets, powders and/or concentrated electrolyte solutions, can be automatically dispensed and mixed with purified water, bicarbonate, and/or sodium chloride in a mixing chamber to produce the dialysate according to the desired dialysate recipe.

Embodiments of the disclosure allow for chemical constituents to be delivered and stored in a tablet form or in a concentrated form, thus requiring minimal storage space and oversight. Mixing the dialysate in batches throughout dialysis suggests less storage space is required since the volume of dialysate made can be fully exhausted during a treatment session.

Embodiments of the disclosure allow for the dialysate composition used during dialysis to be personalized, whereby a patient's individual responses to dialysis are taken into account by monitoring his electrolyte responses to the dialysis treatment throughout the treatment session. In this way, a one-size-fits-all rule or a coarse heuristic is not applied during the treatment. The electrolyte composition monitor, through its continuous adjustments of dialysate composition, can effectively personalize treatment to the individual patient, ensuring that the patient does not leave the dialysis treatment with deficient levels or elevated levels of certain monitored electrolytes, and improving long-term outcomes and patient satisfaction.

Embodiments of the disclosure allow for an electrolyte composition monitor that can, over time, learn a dialysis recipe or formula most appropriate for the patient. By continually adjusting the dialysate in batches, the electrolyte composition monitor can determine which electrolytes the patient is typically sensitive to; thus, in further treatments, the electrolyte composition monitor can suggest a starting dialysate recipe that is more appropriate for the patient. In this way, embodiments of the electrolyte composition monitor will allow for a learning model tailored to adapt to evolving patient needs.

Embodiments of the disclosure provide individualized dialysis treatment based on online monitoring of electrolytes in the patient's blood by generating individualized dialysate as electrolyte conditions in the patient's blood change during treatment. This improvement solves a problem in current treatment practice where dialysate formulas and recipes for individual patients are based on monthly lab blood test results. Dialysis patients are rarely in steady state, so a lab blood test may be outdated by the time the patient enters the clinic for dialysis. Thus, reliance on monthly lab testing may prove harmful or of limited benefit to individual patients.

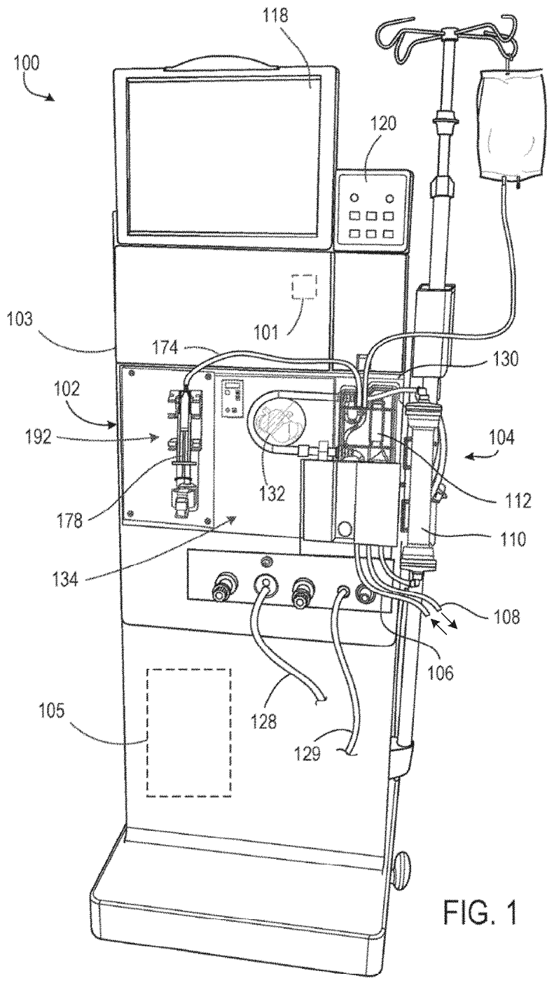

FIG. 1 shows a dialysis system, in particular, a hemodialysis system 100. Although the system described herein is largely described in connection with hemodialysis systems by way of example, it is explicitly noted that the system described herein may be used in connection with other types of medical devices and treatments, including peritoneal dialysis systems. The hemodialysis system 100 includes a hemodialysis machine 102 connected to a disposable blood component set 104 that partially forms a blood circuit. During hemodialysis treatment, an operator connects an arterial patient line 106 and a venous patient line 108 of the blood component set 104 to a patient. The blood component set 104 includes an air release device 112. As a result, if blood passing through the blood circuit during treatment contains air, the air release device 112 will vent the air to atmosphere.

The blood component set 104 is secured to a module 130 attached to the front of the hemodialysis machine 102. The module 130 includes a blood pump 132 capable of circulating blood through the blood circuit. The module 130 also includes various other instruments and sensors, e.g., electrolyte sensors, capable of monitoring the blood flowing through the blood circuit. The module 130 includes a door that when closed, as shown in FIG. 1, cooperates with the front face of the module 130 to form a compartment that is sized and shaped to receive the blood component set 104.

The blood pump 132 is part of a blood pump module 134. The blood pump module 134 includes a display window, a start/stop key, an up key, a down key, a level adjust key, and an arterial pressure port. The display window displays the blood flow rate setting during blood pump operation. The start/stop key starts and stops the blood pump 132. The up and down keys increase and decrease the speed of the blood pump 132. The level adjust key raises a level of fluid in an arterial drip chamber.

The hemodialysis machine 102 further includes a dialysate circuit formed by the dialyzer 110, various other dialysate components, and dialysate lines connected to the hemodialysis machine 102. Many of these dialysate components and dialysate lines are inside the housing 103 of the hemodialysis machine 102 and are thus not visible in FIG. 1. During treatment, while the blood pump 132 circulates blood through the blood circuit, dialysate pumps (not shown) circulate dialysate through the dialysate circuit.

The dialysate is created by the hemodialysis machine 102 in batches. That is, the hemodialysis machine 102 is configured to mix various chemical constituents of the dialysate together to form a dialysate batch having requisite characteristics based on measurements of electrolyte concentration in the patient's blood. In this way, dialysate used during the dialysis treatment can be optimized for the specific patient for different phases of the treatment based on how the patient is responding to the treatment.

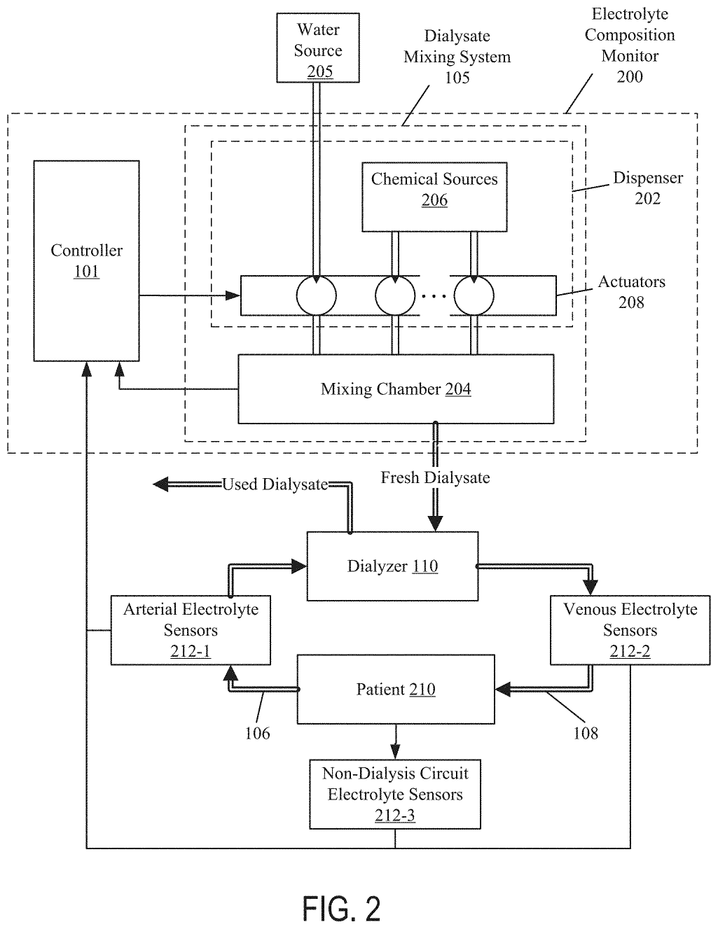

The hemodialysis machine 102 includes an electrolyte composition monitor (200 of FIG. 2), which is made up of a controller 101 and a dialysate mixing system 105 for mixing dialysate. During dialysis, the controller 101 is configured to receive electrolyte measurements from the patient's blood, and the controller 101 is configured to provide signals for adjusting the dialysate recipe for dialysate batches throughout the dialysis treatment. The dialysate mixing system 105 is internal to the housing 103 of the hemodialysis machine 102. In an embodiment, water, sodium chloride (NaCl), bicarbonate (NaHCO3), and a plurality of chemical concentrates are mixed together to form the dialysate. The dialysate mixing system 105 provides already mixed dialysate to the dialyzer 110 via at least a dialysate supply line, which is also internal to the housing 103 of the hemodialysis machine 102. A drain line 128 and an ultrafiltration line 129 extend from the hemodialysis machine 102. The drain line 128 and the ultrafiltration line 129 are fluidly connected to the various dialysate components and dialysate lines inside the housing 103 of the hemodialysis machine 102 that form part of the dialysate circuit. During hemodialysis, the dialysate supply line carries fresh dialysate through various dialysate components, including the dialyzer 110. As the dialysate passes through the dialyzer 110, it collects toxins from the patient's blood. The resulting spent dialysate is carried from the dialysate circuit to a drain via the drain line 128. When ultrafiltration is performed during treatment, a combination of spent dialysate and excess fluid drawn from the patient is carried to the drain via the ultrafiltration line 129.

In an embodiment, the controller 101 determines the chemical composition of each batch of dialysate. For example, a batch of dialysate may be 12 liters (L) and the chemical composition may include a plurality of chemical concentrates. The chemical concentrates may be liquid concentrates of varying viscosity and/or may be solid concentrates in the form of tablets, pills, or powders. The controller 101 may compute the chemical composition (e.g., an amount of each of the plurality of chemical concentrates such as a number of tablets) for each 12 L batch of dialysate based on a prescription issued by a physician/doctor.

In an embodiment, the controller 101 may use a reduced volume of the dialysate for the dialysis treatment of the patient. For example, the controller 101 may reduce the dialysate to blood flow ratio for the dialysis treatment. By reducing the dialysate to blood flow ratio, the dialysis treatment may consume less dialysate (e.g., 40 L of the dialysate per dialysis treatment may be used instead of 120 L).

A drug pump 192 also extends from the front of the hemodialysis machine 102. The drug pump 192 is a syringe pump that includes a clamping mechanism configured to retain a syringe 178 of the blood component set 104. The drug pump 192 includes a stepper motor configured to move the plunger of the syringe 178 along the axis of the syringe 178. The drug pump 192 can thus be used to inject a liquid drug (e.g., heparin) from the syringe 178 into the blood circuit via a drug delivery line 174 during use, or to draw liquid from the blood circuit into the syringe 178 via the drug delivery line 174 during use.

The hemodialysis machine 102 includes a user interface with input devices such as a touch screen 118 and a control panel 120. The touch screen 118 and the control panel 120 allow an operator to input various different treatment parameters to the hemodialysis machine 102 and to otherwise control the hemodialysis machine 102. The touch screen 118 allows an operator to select between user profiles, and the control panel 120 can allow the operator to select between user profiles by scanning the patient's membership card. The touch screen 118 displays information to the operator of the hemodialysis system 100. The controller 101 is also configured to receive and transmit signals to the touch screen 118 and the control panel 120. The controller 101 can control operating parameters of the hemodialysis machine 102, e.g., providing signals at appropriate times for adjusting composition of dialysate throughout a dialysis treatment. The dialysate mixing system can be, e.g., the dialysate mixing system in Kalaskar et al., US 2018/0326138, which is hereby incorporated herein in its entirety.

FIG. 2 is a block diagram illustrating use of an electrolyte composition monitor 200 with a patient 210 during dialysis, according to embodiments of the disclosure. Components of the hemodialysis system 100 of FIG. 1 are used as an example, but as previously stated, the electrolyte composition monitor 200 can be used in peritoneal dialysis. The electrolyte composition monitor 200 is configured to receive electrolyte measurements from one or more electrolyte sensors 212. The electrolyte composition monitor 200 is also configured to use the electrolyte measurements to adjust dialysate recipe, mix a new batch of dialysate, and provide fresh dialysate to the dialyzer 110.

The electrolyte composition monitor 200 includes the controller 101 and the dialysate mixing system 105. The controller 101 is configured to interface with the electrolyte sensors 212 to receive the electrolyte measurements. Examples of the controller 101 include a field programmable gate array (FPGA), an application specific integrated circuit (ASIC), and a processor with non-transitory computer-readable medium.

The dialysate mixing system 105 includes a dispenser 202 and a mixing chamber 204. The dispenser 202 can include chemical concentrates in chemical sources 206. The chemical concentrates are used as ingredients of the dialysate mixture. The chemical concentrates can be liquid concentrates of varying viscosity or can be solid concentrates in the form of tablets, pills, or powders. The chemical sources 206 are containers that hold these chemical concentrates. Chemical sources 206 can thus hold concentrates of potassium chloride (KCl), calcium chloride (CaCl₂), magnesium chloride (MgCl₂), citric acid, dextrose, sodium chloride (NaCl), sodium bicarbonate (NaHCO₃), acetic acid, glucose, and so on. Not all chemical concentrates available need to be used in a dialysate formula or recipe. For example, one recipe may only call for acetic acid, NaCl, CaCl₂, KCl, MgCl₂, and glucose. Another recipe may call for bicarbonate, NaCl, CaCl₂, KCl, and MgCl₂, without glucose.

The dispenser 202 can further include actuators 208 that aid in dispensing specific amounts of the chemical concentrates to a mixing chamber 204 for mixing a batch of dialysate. The actuators 208 not only control the amount of chemical concentrates provided to the mixing chamber 204, but also control an amount of water used in mixing the batch of dialysate. A water source 205 can be a water connection for receiving filtered water or water suitable for use in dialysis treatment. The water source 205 can connect to the hemodialysis system 100 via an inlet tube.

The dispenser 202 provides the chemical concentrates and water to the mixing chamber 204. Contents in the mixing chamber 204 are agitated for an appropriate amount of time until chemical concentrates are sufficiently distributed throughout. In some embodiments, the mixing chamber 204 increases its temperature to assist in dissolving and/or distributing the chemical concentrates to yield a homogenous solution. After realizing a homogenous solution, the mixing chamber 204 can be brought to an appropriate temperature for dialysis treatment.

The mixing chamber 204 of the dialysate mixing system 105 provides the mixed dialysate to the dialyzer 110. In an embodiment, the mixing chamber 204 is multi-chambered where a first chamber is used for mixing dialysate and a second chamber is used for storing and delivering dialysate to the dialyzer 110. In an embodiment, the mixing chamber 204 includes sensors for sensing levels of dialysate in both the first and second chambers. The mixing chamber 204 may also alert the controller 101 when a batch of dialysate has been mixed and when fresh dialyzer from the batch of dialysate is provided to the dialyzer 110.

The dialyzer 110 receives blood from the patient 210 via the arterial patient line 106. Electrolyte sensors along the arterial patient line 106 may be provided for measuring electrolyte concentration in blood upstream of the dialyzer 110. These electrolyte sensors are identified as arterial electrolyte sensors 212-1 in FIG. 2. The dialyzer 110 returns blood to the patient 210 via the venous patient line 108. Electrolyte sensors along the venous patient line 108 may be provided for measuring electrolyte concentration in blood downstream of the dialyzer 110. These electrolyte sensors can be identified as venous electrolyte sensors 212-2. Furthermore, electrolyte sensors not interrupting the dialysis circuit can be interfaced with the patient 210. For example, a sensor can be placed along a peripherally inserted central catheter (PICC) line to measure electrolyte concentration. These sensors are identified as non-dialysis circuit electrolyte sensors 212-3.

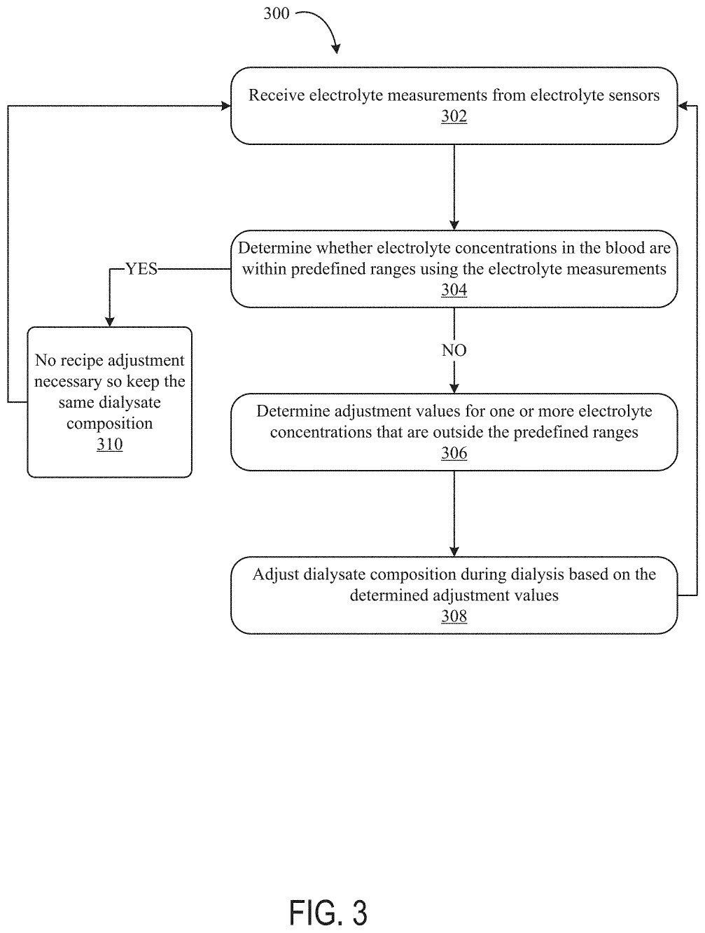

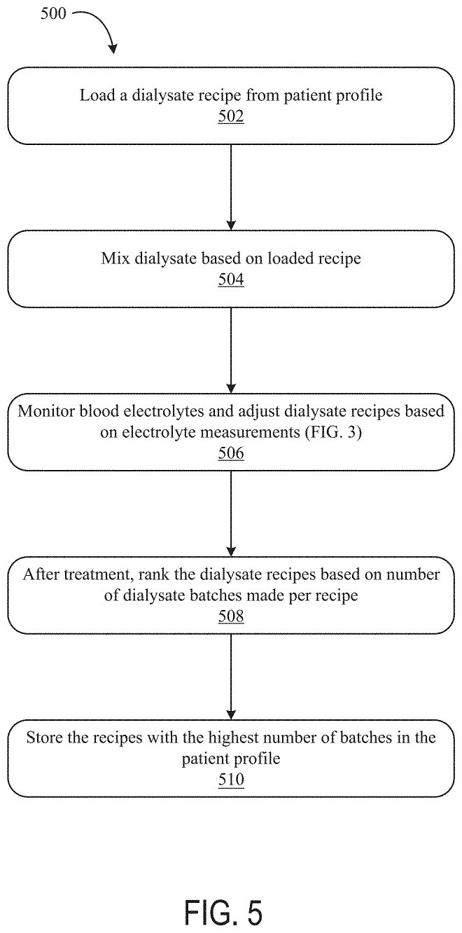

The electrolyte sensors 212 are configured to measure electrolyte concentration in the blood of the patient 210. The electrolyte sensors 212 can be, for example, conductivity sensors, nuclear magnetic resonance (NMR) sensors and/or optical sensors. NMR sensors may detect, determine, and/or obtain real-time electrolyte concentrations (e.g., sodium concentrations) in the blood of the patient 210. Additionally, and/or alternatively, NMR sensors may be modified (e.g., re-tuned to different radio frequencies) to detect, determine, and/or obtain real-time potassium and/or phosphorous concentrations. Additionally, and/or alternatively, in some embodiments, an NMR sensor measures concentrations of free sodium in both the dialysate and the blood, each sampled separately, and the concentration of free sodium is reported to the controller 101. Furthermore, the sodium and other electrolyte concentrations may vary from patient to patient and even for a given patient between consecutive dialysis sessions. Accordingly, using an NMR sensor or other sensor to measure these concentrations in real-time to adjust the electrolyte concentrations and even using individualized recipes (described in FIGS. 3 and 5) may be beneficial to provide the optimal treatment for the patient during dialysis. Additionally, and/or alternatively, the electrolyte sensors 212 may include optical sensors configured to detect real-time electrolyte concentrations such as calcium concentrations and/or magnesium concentrations.

Examples of an NMR sensor usable with exemplary embodiments of the present application are described in further detail in U.S. Pat. No. 10,371,775 (Titled: Dialysis System With Radio Frequency Device Within A Magnet Assembly For Medical Fluid Sensing And Concentration Determination), granted on Aug. 6, 2019, and U.S. Provisional Patent Application No. 62/967,349 (Titled: Individualized And On-Demand Dialysis System With Networking Capabilities), filed on Jan. 29, 2019, both of which are incorporated by reference herein in its entirety.

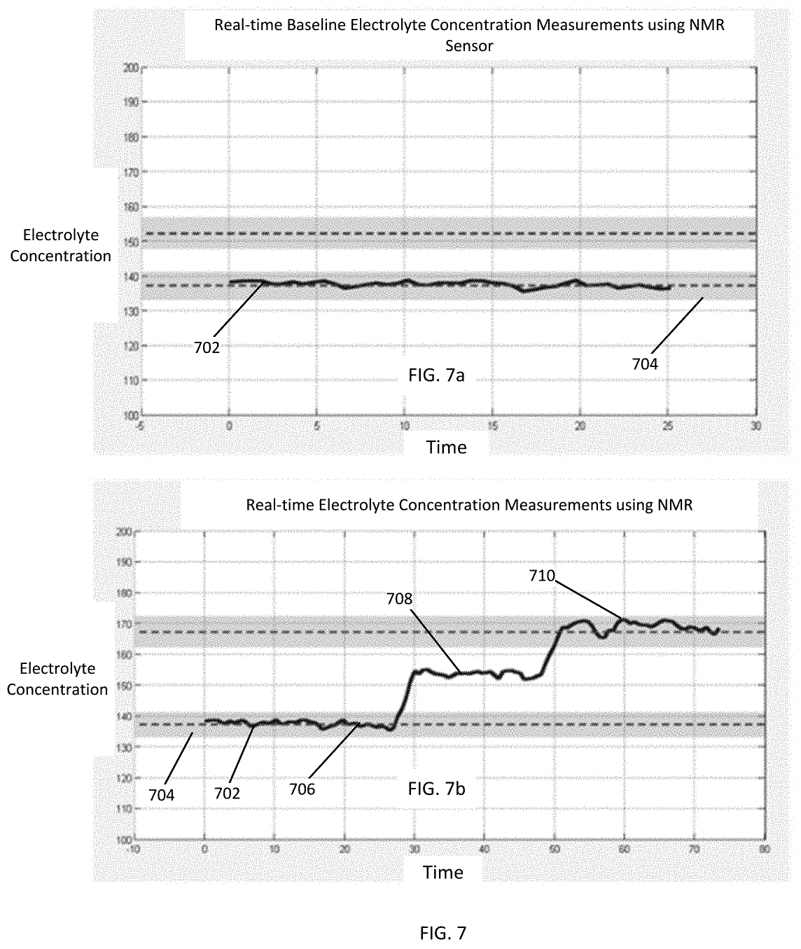

Furthermore, FIG. 7 shows graphical representations of real-time measurements obtained using an NMR sensor. For example, FIG. 7a shows real-time electrolyte concentration (e.g., sodium concentration) measurements using the NMR sensor. Line 702 indicates the sodium concentration and the shaded area 704 represents the accuracy margin. FIG. 7b also shows real-time electrolyte concentration measurements using the NMR sensor. For example, portion 704 of line 702 indicates the baseline sodium concentration. Then, portion 708 indicates a first adjustment of the baseline sodium concentration (e.g., introducing or injecting sodium boluses to increase the sodium concentration). Portion 710 shows another injection of sodium boluses for increasing the sodium concentration again.

FIG. 3 is a flow diagram for managing electrolytes in blood of a dialysis patient during dialysis, according to an embodiment of the disclosure. FIG. 3 is a flow diagram illustrating a process 300 that an electrolyte composition monitor, e.g., electrolyte composition monitor 200, can perform in managing electrolytes in blood of patient 210. At 302, the controller 101 of the electrolyte composition monitor 200 receives (e.g., obtains) electrolyte measurements from electrolyte sensors 212. The obtained electrolyte measurements may include sodium, potassium, phosphorous, magnesium, and/or calcium electrolyte concentrations in the blood of the patient 210.

At 304, the controller 101 determines from the electrolyte measurements whether electrolyte concentrations in the blood are within predefined ranges. In an example, electrolyte concentration of sodium in the blood should be within 135-145 mEq/L range, electrolyte concentration of potassium should be within 3.5-5 mEq/L range, electrolyte concentration of calcium should be within 8.5-10.2 mg/dL (2-2.6 mmol/L) range, and so on. The electrolyte measurements received at the controller 101 are prepared in a manner to obtain electrolyte concentrations. For example, if a sodium NMR sensor provides radio frequency (RF) energy level at a resonant frequency of sodium as measurement signals, then the controller 101 analyzes the RF energy level provided to determine the concentration of sodium in the blood. This concentration of sodium is compared to the upper and lower bounds of the predefined range for sodium to determine whether sodium concentration in the blood is within the predefined range.

In some examples, the predefined ranges are clinically defined ranges such as clinically known ranges. In other examples, the predefined ranges may be individualized. For example, as described below in 502 of FIG. 5, the dialysate recipe is a recipe determined based on historical trend analysis on electrolyte measurements from the patient's previous dialysis treatments. In other words, the dialysate recipe is individualized for the patient 210 based on the previous dialysis treatments performed on the patient 210. The dialysate recipe associated with the patient may include electrolyte ranges (e.g., an electrolyte concentration range for sodium, potassium, calcium, magnesium, and/or phosphorous). Further, as described below in FIG. 5, the controller 101 may load the dialysate recipe prior to beginning the dialysis treatment. At 302, the controller 101 determines the predefined ranges based on the loaded dialysate recipe and may compare these predefined ranges with the electrolyte concentrations from the electrolyte measurements.

At 306, the controller 101 determines adjustment values, based on the plurality of electrolyte measurements, for one or more electrolyte concentrations outside the predefined ranges. The controller 101 determines, for each electrolyte concentration outside of the predefined ranges, whether to increase or decrease concentration of the electrolyte. Increasing or decreasing the concentration provides directionality to the adjustment values. The controller 101 then determines the magnitude of the adjustment value by determining a target amount by which the concentration of the electrolyte should be increased.

In an embodiment, the controller 101 determines adjustment values by unit increments. That is, after determining whether to increase or decrease a concentration of an electrolyte that is not within a predefined range, the controller 101 determines that the concentration of the electrolyte should be adjusted by a given unit. In an embodiment where chemical constituents of dialysate are adjusted by tablets, each unit represents an electrolyte concentration provided by a chemical concentrate's respective tablet. In an embodiment where chemical constituents of dialysate are adjusted by liquid concentrates, each unit represents an expected electrolyte concentration provided by opening its respective valve for a predetermined amount of time. Although one unit increments are described, adjustment values can be determined as multiple unit increments. For example, the controller 101 can determine that the concentration of the electrolyte that is not within its predefined range should be increased by three units which correspond to an amount of electrolytes expected from three tablets.

In an embodiment, the controller 101 determines adjustment values based on pre-programmed dialysate recipes, formulas or prescriptions. The controller 101 can store one or more recipes for various electrolyte conditions in its memory. For example, the memory may include a recipe for low sodium, high sodium, low potassium, high potassium, and so on. Each of these dialysate recipes can be tagged as being effective in reducing or raising one or more electrolyte concentrations. That way, based on a combination of electrolytes determined to be outside their respective predefined ranges, the controller 101 can select a recipe from one of these predefined recipes for the next batch of dialysate.

In some examples and referring to FIG. 5 and process 500 below, the controller 101 determines the magnitude and/or directionality of the adjustment values (e.g., unit increments) based on the loaded dialysate recipe from 502. For example, the controller 101 may determine and load the dialysate recipe based on historical trend analysis on electrolyte measurements from the patient's previous dialysis treatments (e.g., based on the most effective recipe from the historical trend analysis, the dialysate recipe with the greatest number of batches in the patient profile, and/or the most recent dialysate recipe used during the dialysis treatment). For instance, if the high sodium recipe has the greatest number of made batches in the patient profile (e.g., the dialysate solution was created/adjusted the greatest number of times using the recipe), the controller 101 may determine the high sodium recipe as the most effective dialysate recipe and load that recipe at 502. Then, at 306, the controller 101 may determine the magnitude of the adjustment values using this recipe.

In an embodiment, the controller 101 determines that one or more electrolyte concentrations outside the predefined ranges deviates significantly from the predefined ranges. For example, at 304, a potassium concentration of 6.0 mEq/L is determined, and the predefined range for potassium is between 3.5 and 5.0 mEq/L. The potassium concentration is then determined by the controller 101 to be too high. The controller 101 can determine that the next dialysate batch should decrease the potassium concentration. Thus, the controller 101 can determine an adjustment value for potassium that reduces the potassium ion concentration in the next batch of dialysate as prescribed. Although potassium is used as an example, the controller 101 can determine that concentration of more than one electrolyte in the blood is too high and determine adjustment values to make a next batch of dialysate. In other words, the controller 101 may determine that an electrolyte concentration (e.g., potassium) is outside of the predefined ranges and determine one or more adjustment values for the next dialysate batch. The one or more adjustment values may be a single adjustment value for the electrolyte concentration (e.g., potassium) or may include multiple adjustments values for multiple different electrolyte concentrations (e.g., potassium, calcium, and so on).

Additionally, and/or alternatively, the controller 101 may determine multiple electrolyte concentrations (e.g., potassium and calcium) are outside of the predefined ranges and may determine one or more adjustment values for the next dialysate batch. The one or more adjustment values may be a single adjustment value for the electrolyte concentration (e.g., potassium) or may include multiple adjustments values for multiple different electrolyte concentrations (e.g., calcium, potassium, and so on).

In an embodiment, the controller 101 determines that a majority or all of the electrolyte concentrations are outside the predefined ranges and adjustment values of all the electrolyte concentrations have a same direction. The controller 101 can determine adjustment values based on the amounts of chemicals supplied by the chemical sources 206 and the amount of water to include in the dialysate. In some instances, each batch of the dialysate may be 12 L. In other instances, the batches may be greater than 12 L such as 24 L. The controller 101 can determine adjustment values based on the amount of chemicals supplied by the chemical sources 206, the amount of water to include in the dialysate, and the volume of the batch of the dialysate. For instance, if the prescription indicates that 2 potassium tablets are used for a 12 L batch, the controller 101 may determine to use 4 potassium tablets for a 24 L batch.

At 308, the controller 101 provides instructions to the dispenser 202 to adjust the composition of the dialysate during dialysis based on the determined adjustment values of 306. The composition of the dialysate includes chemicals from the chemical sources 206. In an embodiment, the controller 101 generates adjustment signals for changing the composition of the dialysate during dialysis based on the determined adjustment values. The controller 101 then provides actuating signals to actuators 208 for changing how much of each chemical concentrate to release into the mixing chamber 204. By effecting a change in an amount of any of the chemical concentrates released into the mixing chamber 204, the controller 101 causes the dispenser 202 to change proportions of the chemicals in the dialysate.

In an embodiment, when a respective adjustment value for an electrolyte concentration outside the respective predefined range indicates an increase, the controller 101 generates a respective adjustment signal for dispensing a higher proportion of a respective chemical, thus increasing a chemical contribution of a respective chemical source in the chemical sources 206. When the respective adjustment value for the electrolyte concentration outside the respective predefined range is a decrease, the controller 101 generates a respective adjustment signal for dispensing a lower proportion of the respective chemical of the respective chemical source in the chemical sources 206.

In an embodiment, the adjustment signals the controller 101 provides to the dispenser 202 are encoded as a number of electrical pulses. Electrical pulses can be voltage or current pulses. For example, a number of pulses provided by the controller 101 to a respective actuator in the actuators 208 can encode an amount of a respective chemical in the chemical sources 206 to release into the mixing chamber 204. In a previous dialysate batch, if 5 pulses were provided to an actuator that controls a release of CaCl₂ tablets into the mixing chamber 204, then for a next dialysate batch, if 4 pulses are provided to the actuator then a lower number of CaCl₂ tablets will be released into the mixing chamber 204. Thus, the adjustment signals generated by the controller 101 can be encoded as a change in a number of electrical pulses provided to one or more actuators. The change in number of electrical pulses can be an increase in the number of electrical pulses or a decrease in the number of electrical pulses. Furthermore, all but one actuator in the actuators 208 can receive a reduced number of electrical pulses. Conversely, all but one actuator in the actuators 208 can receive an increased number of electrical pulses.

After completing 308, the controller 101 cycles back to 302 and receives new electrolyte measurements (e.g., second electrolyte measurements from the sensors 212). The process 300 is performed by the electrolyte composition monitor 200 until the dialysis treatment of patient 210 ends.

For example, in subsequent iterations, at 304, the controller 101 determines from the electrolyte measurements whether the first adjustment values caused the new electrolyte measurements to be within the predefined ranges. If no electrolyte concentrations in the blood are outside the predefined ranges, then the controller 101 determines at 310 that no adjustment is necessary. The controller 101 keeps the most recent recipe for the new dialysate batch and the process 300 returns to 302. If there are electrolyte concentrations that are still outside of the predefined ranges, the controller 101 may determine new adjustment values based on the recipe and provide additional instructions to adjust the composition of the dialysate during dialysis. Furthermore, the controller 101 may determine the effectiveness of the previous recipe used and/or determine a new recipe to use for the adjustment values.

For example, in some instances, the controller 101 may determine the effectiveness of the recipe using process 300. As described above, the controller 101 may determine the directionality and/or magnitude of the adjustment values based on the loaded recipe. For example, the controller 101 may obtain a first and a second electrolyte measurement from the electrolyte sensors 212. The first electrolyte measurement may be obtained in the first iteration of process 300 and the second electrolyte measurement may be obtained in the second iteration of process 300 (e.g., the second electrolyte measurement may be subsequent to adjusting the composition of the dialysate during dialysis). The controller 101 may compare the first electrolyte measurement, the second electrolyte measurements, and/or the predefined ranges to determine the effectiveness of the recipe. For instance, if the electrolyte concentration is within the predefined ranges after the adjustment, the controller 101 may determine the recipe used for the adjustment values at 306 is effective. If the electrolyte concentration is still not within the predefined ranges, the controller 101 may determine the recipe is not effective.

Additionally, and/or alternatively, the controller 101 may determine the effectiveness of the recipe based on how close the second electrolyte measurement is to the predefined range. For instance, if the second electrolyte measurement is within the predefined range, the controller 101 may determine the recipe is very effective. If the second electrolyte measurement is close to the predefined range, but is not within the predefined range it, the controller 101 may determine the recipe is effective. If the second electrolyte measurement is not close to the predefined range, the controller 101 may determine the recipe is not effective. If the second electrolyte measurement is even further away from the predefined range compared to the first electrolyte measurement, the controller 101 may determine the recipe is extremely ineffective.

In some variations, the controller 101 may dynamically rank recipes during the dialysis treatment (e.g., during process 300). For example, after creating each batch of the dialysate solution using the recipe, the controller 101 may determine the effectiveness of the recipe. Then, the controller 101 may determine whether to load a new dialysate recipe based on the updated effectiveness of the recipe. If the controller 101 loads a new dialysate recipe, the controller 101 may use the new dialysate recipe to determine the adjustment values. In other words, during the dialysis treatment, the controller 101 may use multiple different recipes to determine the adjustment values based on the determined effectiveness of the recipes during the treatment of the patient.

In some instances, the controller 101 may rank the recipes after the dialysis treatment for the patient has concluded (e.g., after process 300 has concluded). For example, the controller 101 may determine the effectiveness of the one or more recipes used during the dialysis treatment based on comparing the electrolyte concentration after the adjustment with the predefined ranges. Then, the controller 101 may store the associated effectiveness of the recipes in memory and/or rank the recipes based on the effectiveness. The next time the patient undergoes dialysis treatment, the controller 101 may load the highest ranking stored recipe for the predefined ranges and/or the adjustment values.

In some examples, process 300 may be used for peritoneal dialysis (PD solutions). For peritoneal dialysis, process may further include a sterilization step. For example, prior to 308, the controller 101 may provide instructions to the dispenser 202 to sterilize the composition of the dialysate including the chemicals from the chemical sources 206. Then, at 308, the controller 101 provides instructions to the dispenser 202 to sterilize the chemicals from the chemical sources.

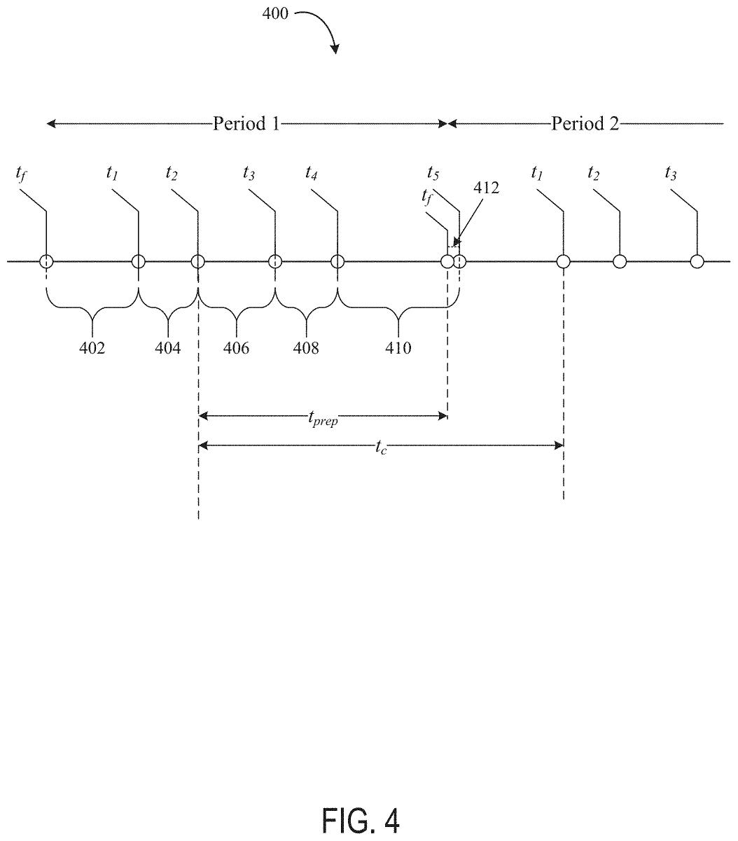

FIG. 4 illustrates an example timeline 400 for managing electrolytes in blood of a dialysis patient during dialysis. As described above with respect to FIG. 3, process 300 is cyclic or periodic, so with respect to the timeline 400, one period of activities is highlighted via timestamps t_{f}, t₁, t₂, t₃, t₄, and t₅. The timestamps are defined as follows:
t_{f}—Time when the controller 101 receives a fresh dialysate signal indicating that a new batch of dialysate is mixed and ready for use
t₁—Time when the controller 101 receives electrolyte measurements from the electrolyte sensors 212
t₂—Time when the controller 101 sends adjustment signals to the actuators 208 of the dispenser 202
t₃—Time when the actuators 208 allow chemicals and water to migrate from the chemical sources 206 and water source 205, respectively, to the mixing chamber 204
t₄—Time when the mixing chamber 204 starts mixing the new batch of dialysate
t₅—Time when an old batch of dialysate is depleted

FIG. 4 organizes activities in FIG. 3 according to the example timeline 400. In Period 1, at the start of dialysis, a fresh batch of dialysate is mixed and ready for use. At this point, the mixing chamber 204 provides a fresh dialysate signal to the controller 101 at timestamp t_{f}. After a time duration 402, the controller 101 receives, at timestamp t₁, electrolyte measurements from the electrolyte sensors 212. During a time duration 404, the controller 101 determines adjustment signals to provide to the actuators 208, and at timestamp t₂, sends the adjustment signals to the actuators 208. The actuators 208 respond to the adjustment signals after a time duration 406, so at timestamp t₃, the actuators 208 allow chemicals and water to migrate from their respective sources into the mixing chamber 204. After a time duration 408, the mixing chamber 204 then mixes its contents, at timestamp t₄, to form a new batch of dialysate.

At timestamp t₅, the old batch of dialysate is completely depleted from the mixing chamber 204, so time duration 410 indicates a time between when the mixing chamber 204 begins mixing contents for the new batch of dialysate and when the old batch of dialysate is depleted. In some embodiments, an error is not generated by the controller 101 when the new batch of dialysate is ready before the old batch of dialysate is depleted. This condition is indicated in FIG. 4 by showing that a fresh dialysate signal is provided at timestamp t_{f} during time duration 410.

In an embodiment, the controller 101 can optimize the process 300 by trying to reduce the time duration 412 between timestamps t_{f} and t₅. That way, the new batch of dialysate is ready at a same time that the old batch of dialysate is depleted so that when the fresh dialysate signal is received at the controller 101, the controller 101 can determine an appropriate time duration 402 to wait before obtaining electrolyte measurements from the electrolyte sensors 212. That way, the controller 101 gives enough time to be able to view the effects of the new batch of dialysate on the electrolytes in the blood.

Put another way, the controller 101 can monitor tₚᵣₑₚ, a time duration between when the controller 101 sends adjustment signals to the actuators 208 and when the controller 101 receives the fresh dialysate signal from the mixing chamber 204. The controller 101 can try to optimize tₚᵣₑₚ such that its duration is substantially the same as the sum of durations 406, 408, and 410.

In an embodiment, the controller 101 determines that if an adjustment signal is sent at a certain time, then there would be a violation of tₚᵣₑₚ, that is, timestamp t₅ would be reached before the new batch of dialysate is mixed and ready. The controller 101 can determine in this case to delay the adjustment signal, mix a new batch of dialysate based on an old recipe, and then provide the buffered adjustment signal in a next period. This indicates that after timestamp t_{f}, there is a maximum time tₘₐₓ that the controller 101 can wait before sending the adjustment signals to the actuators 208 at timestamp t₂. In an embodiment tₘₐₓ can be determined to be the sum of durations 402, 404, 406, 408, and 410 minus tₚᵣₑₚ. Since tₘₐₓ depends on timestamp t₅, in some embodiments, tₘₐₓ is determined by the controller 101 based on flow rate of dialysate exiting the mixing chamber 204 and a volume of dialysate in the mixing chamber 204.

In an embodiment, the controller 101 can also monitor and try to regularize t_{c}, a time duration between when the controller 101 sends an adjustment signal and when the controller 101 obtains electrolyte measurements to ascertain effects of the adjustment signals on electrolyte concentration in the blood.

FIG. 5 is a flow diagram for determining individualized dialysate recipes or prescriptions for a patient, according to an embodiment of the disclosure. FIG. 5 is a flow diagram illustrating a process 500 performed by a dialysis system, e.g., the hemodialysis system 100, to determine the patient's dialysate recipes. At 502, the hemodialysis system 100 loads a dialysate recipe from a patient profile.

In an embodiment, the hemodialysis system 100 may receive a chip card or a computer memory storage like a flash drive that contains dialysate recipes for the patient 210. In an embodiment, the patient profile may be obtained from a database or centralized storage. By way of example, for a description of a system for securely distributing information, including medical prescriptions, within a connected health network, reference is made to US Pub. No. 2018/0316505A1 to Cohen et al., which is incorporated herein by reference.

The dialysate recipe for treatment is selected and loaded from the patient profile. In an embodiment, the dialysate recipe selected is a last recipe used from a previous treatment that the patient 210 went through. In another example, the dialysate recipe selected is a default recipe especially when the patient 210 has never undergone dialysis at the specific location. In another example, the dialysate recipe selected is a recipe determined based on trend analysis of previous dialysate recipes from the patient profile. In another example, the dialysate recipe selected is a recipe determined based on historical trend analysis on electrolyte measurements from the patient's previous dialysis treatments.

At 504, the hemodialysis system 100, via the dialysate mixing system 105, mixes a first batch of dialysate based on the loaded recipe from 502.

At 506, the hemodialysis system 100 via the electrolyte composition monitor 200, monitors blood electrolytes and adjusts dialysate recipes based on electrolyte measurements according to various embodiments of the disclosure. For example, the electrolyte composition monitor 200 monitors blood electrolytes and adjusts dialysate recipes as provided in the process 300. During treatment, the hemodialysis system 100 creates a folder or a collection of dialysate entries within the patient profile for the current dialysis treatment. Within the folder, the hemodialysis system 100 can store one or more of dialysate recipe used, number of batches mixed that correspond to the dialysate recipe, and electrolyte measurements that led the dialysate recipe.

At 508, after the dialysis treatment is completed, the hemodialysis system 100 ranks the dialysate recipes stored at 506. In an embodiment, the dialysate recipes are ranked based on a number of dialysate batches made per recipe. In other words, if the hemodialysis system 100 determines the dialysate batches are effective (e.g., effective in reducing the electrolyte concentration(s) to the predefined range in 304), the hemodialysis system 100 may use the recipe again, which would increase the number of dialysate batches made using the recipe and would cause the hemodialysis system 100 to rank the dialysate recipe higher. In an embodiment, the dialysate recipes are ranked based on a trend analysis that compares similar dialysate recipes, then combines the number of batches for the similar dialysate recipes, and then ranks groups of dialysate recipes based on the combined number of batches.

In an embodiment, the similar dialysate recipes with the highest combined number of batches are analyzed to determine one representative recipe. The representative recipe can be determined via one or more statistical means, e.g., can be determined using an average, a median, a random selection, and so on.

At 510, the hemodialysis system 100 stores the dialysate recipes with the highest number of batches in the patient profile. In an embodiment, a representative recipe determined according to embodiments of the disclosure is stored along with the dialysate recipes.

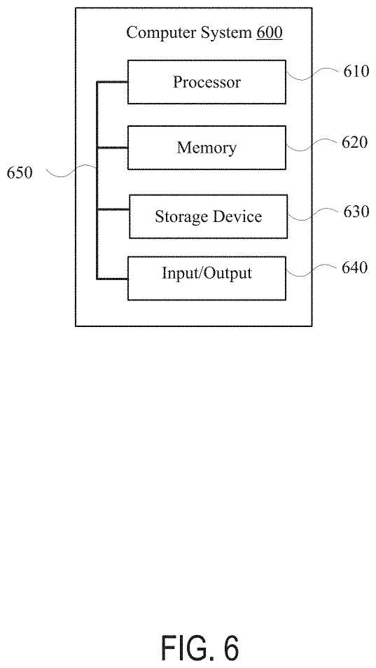

FIG. 6 is a block diagram of an example computer system 600. For example, the controller 101 is an example of the system 600 described here. The system 600 includes a processor 610, a memory 620, a storage device 630, and an input/output device 640. Each of the components 610, 620, 630, and 640 can be interconnected, for example, using a system bus 650. The processor 610 processes instructions for execution within the system 600. The processor 610 can be a single-threaded processor, a multi-threaded processor, or a quantum computer. The processor 610 can process instructions stored in the memory 620 or on the storage device 630. The processor 610 may execute operations that facilitate performing functions attributed to the electrolyte composition monitor 200.

The memory 620 stores information within the system 600. In some implementations, the memory 620 is a computer-readable medium. The memory 620 can, for example, be a volatile memory like synchronous random access memory (SRAM) or a non-volatile memory like flash.

The storage device 630 is capable of providing mass storage for the system 600. In some implementations, the storage device 630 is a non-transitory computer-readable medium. The storage device 630 can include, for example, a hard disk device, an optical disk device, a solid-date drive, a flash drive, magnetic tape, or some other large capacity storage device. The storage device 630 may alternatively be a cloud storage device, e.g., a logical storage device including multiple physical storage devices distributed on a network and accessed using a network. In some implementations, the information stored on the memory 620 can also be stored on the storage device 630.

The input/output device 640 provides input/output operations for the system 600. In some implementations, the input/output device 640 includes one or more of network interface devices (e.g., an Ethernet card), a serial communication device (e.g., an RS-232 10 port), and/or a wireless interface device (e.g., a short-range wireless communication device, an 802.11 card, a 3G wireless modem, or a 4G wireless modem). In some implementations, the input/output device 640 includes driver devices configured to receive input data and send output data to other input/output devices, e.g., a keyboard, a printer, and display devices (such as the touch screen 118). In some implementations, the input/output device 640 receives dialysate prescription (e.g., wirelessly) for processing by the hemodialysis system 100. In some implementations, mobile computing devices, mobile communication devices, and other devices are used for sending dialysate prescriptions.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for adjustment of a dialysate during dialysis for a patient, the method comprising:
subsequent to initiating dialysis for the patient, obtaining, by a controller and from an electrolyte sensor, a measurement of a concentration of an electrolyte in the patient's blood;
determining, by the controller, whether the obtained measurement is within a predefined range;
in response to determining that the measurement is not within the predefined range, determining, by the controller and based on the obtained measurement, at least one first adjustment value for adjusting a composition of the dialysate, wherein the composition of the dialysate is based on respective amounts of chemicals dispensed from a plurality of chemical sources; and
controlling, by the controller and based on the at least one first adjustment value, a dispenser to adjust the composition of the dialysate during dialysis for the patient by changing one or more of the respective amounts of chemicals dispensed from the plurality of chemical sources.

2. The method according to claim 1, wherein controlling, based on the at least one first adjustment value, the dispenser to adjust the composition of the dialysate comprises providing one or more first instructions to direct the dispenser to adjust the composition of the dialysate, and wherein the method further comprises:
subsequent to adjusting the composition of the dialysate during dialysis based on the at least one first adjustment value, obtaining, by the controller, a second measurement of the concentration of the electrolyte in the patient's blood from the one or more electrolyte sensors;
determining, by the controller, whether the at least one first adjustment value caused the second measurement to be within the predefined range; and
in response to determining that the second measurement is not within the predefined range, providing one or more second instructions to the dispenser to adjust the composition of the dialysate during dialysis based on the second measurement.

3. The method according to claim 2, further comprising:
in response to determining that the second measurement is within the predefined range, maintaining the composition of the dialysate during dialysis.

4. The method of claim 1, further comprising:
obtaining, by the controller, a second measurement of a second concentration of a second electrolyte in the patient's blood, wherein the second electrolyte and the first electrolyte are different electrolytes;
determining, by the controller, whether the second measurement is within a second predefined range; and
in response to determining that the second measurement is not within the second predefined range, determining, by the controller and based on the second measurement, at least one second adjustment value for adjusting the composition of the dialysate,
wherein controlling the dispenser to adjust the composition of the dialysate is based on the at least one first adjustment value and the at least one second adjustment value.

5. The method according to claim 1, wherein controlling the dispenser to adjust the composition of the dialysate comprises:
generating, by the controller, actuating signals for changing the composition of the dialysate during dialysis based on the at least one first adjustment value; and
providing, by the controller, the actuating signals to one or more actuators of the dispenser to change proportions of the respective amounts of chemicals dispensed from the plurality of chemical sources.

6. The method according to claim 5, wherein generating the actuating signals comprises:
based on determining the at least one first adjustment value the electrolyte is an increase, generating a first actuating signal for dispensing a higher proportion of a respective chemical of a respective chemical source of the plurality of chemical sources; and
based on determining the at least one first adjustment value the electrolyte is a decrease, generating a second actuating signal for dispensing a lower proportion of the respective chemical of the respective chemical source of the plurality of chemical sources.

7. The method according to claim 5, wherein each actuating signal of the actuating signals is encoded as:
a reduction in the number of electrical pulses provided to one actuator of the one or more actuators,
an increase in the number of electrical pulses provided to one actuator of the one or more actuators,
a reduction in the number of electrical pulses provided to all but one actuator of the one or more actuators, or
an increase in the number of electrical pulses provided to all but one actuator of the one or more actuators.

8. The method according to claim 1, further comprising:
receiving, by the controller, a fresh dialysate signal at time t_{f}, the fresh dialysate signal indicating that the dialysate is mixed and ready for use; and
determining, by the controller and based on a flowrate of the dialysate and a volume of the dialysate, a time tₘₐₓ indicating a maximum amount of time after t_{f} to adjust the composition of the dialysate.

9. The method according to claim 1, wherein the electrolyte sensor is an optical sensor.

10. The method according to claim 9, wherein the electrolyte sensor is the NMR sensor, and wherein the NMR sensor is configured to obtain a real-time sodium concentration, a real-time potassium concentration, or a real-time phosphorous concentration.

11. The method according to claim 1, wherein the electrolyte sensor is located upstream of a dialyzer and configured to interface with a tubing upstream of the dialyzer.

12. The method according to claim 1, wherein the electrolyte sensor is located downstream of a dialyzer and configured to interface with a tubing downstream of the dialyzer.

13. The method according to claim 1, further comprising:
determining a dialysate recipe based on the patient, and
wherein determining the at least one first adjustment value for adjusting the composition of the dialysate is based on the dialysate recipe.

14. The method according to claim 13, wherein determining the dialysate recipe is based on historical trend analysis from the patient's previous dialysis treatments.

15. The method according to claim 13, wherein controlling the dispenser to adjust the composition of the dialysate comprises providing one or more first instructions to the dispenser to adjust the composition of the dialysate, wherein the method further comprises:
subsequent to providing the one or more first instructions, obtaining, by the controller, a second measurement of the concentration of the electrolyte from the one or more electrolyte sensors; and
determining an effectiveness of the dialysate recipe based on the second measurement.

16. The method according to claim 15, wherein the determining the effectiveness of the dialysate recipe is based on whether the second measurement is within the predefined range, and wherein the method further comprises:
based on determining the second measurement is not within the predefined range, selecting a new dialysate recipe;
determining at least one second adjustment value for adjusting the composition of the dialysate based on the new dialysate recipe; and
providing, to the dispenser of the electrolyte composition monitor, one or more second instructions to adjust the composition of the dialysate during dialysis based on the at least one second adjustment value.

17. The method according to claim 15, further comprising:
based on determining the dialysis for the dialysis patient has concluded, storing the dialysate recipe and the determined effectiveness of the dialysate recipe in memory.

18. A non-transitory computer-readable medium having processor-executable instructions stored thereon for adjustment of a dialysate during dialysis for a patient, wherein the processor-executable instructions, when executed, facilitate:
subsequent to initiating dialysis for the patient, obtaining, from an electrolyte sensor, a measurement of a concentration of an electrolyte in the patient's blood;
determining whether the obtained measurement is within a predefined range;
in response to determining that the measurement is not within the predefined range, determining, based on the obtained measurement, at least one first adjustment value for adjusting a composition of the dialysate, wherein the composition of the dialysate is based on respective amounts of chemicals dispensed from a plurality of chemical sources; and
controlling, based on the at least one first adjustment value, a dispenser to adjust the composition of the dialysate during dialysis for the patient by changing one or more of the respective amounts of chemicals dispensed from the plurality of chemical sources.

19. A system for adjustment of a dialysate during dialysis for a patient, comprising:
an electrolyte sensor configured to measurement a measurement of a concentration of an electrolyte in the patient's blood; and
an electrolyte composition monitor, comprising:
a controller, configured to:
subsequent to initiating dialysis for the patient, obtain, from the electrolyte sensor, the measurement of the concentration of the electrolyte in the patient's blood;
determine whether the obtained measurement is within a predefined range;
in response to determining that the measurement is not within the predefined range, determine, based on the obtained measurement, at least one first adjustment value for adjusting a composition of the dialysate, wherein the composition of the dialysate is based on respective amounts of chemicals dispensed from a plurality of chemical sources; and
provide, based on the at least one first adjustment value, one or more first instructions to a dispenser to adjust the composition of the dialysate during dialysis for the patient by changing one or more of the respective amounts of chemicals dispensed from the plurality of chemical sources; and
the dispenser configured to:
adjust the composition of the dialysate during dialysis based on the one or more first instructions.

20. The system according to claim 19, wherein the controller is further configured to:
subsequent to adjusting the composition of the dialysate during dialysis based on the at least one first adjustment value, obtain a second measurement of the concentration of the electrolyte in the patient's blood from the one or more electrolyte sensors;
determine whether the at least one first adjustment value caused the second measurement to be within the predefined range; and
in response to determining that the second measurement is not within the predefined range, provide one or more second instructions to the dispenser to adjust the composition of the dialysate during dialysis based on the second measurement.
